(19) United States
(12) Patent Application Publication
(10) Pub. No.: US 2014/0323734 A1
(43) Pub. Date: Oct. 30, 2014

(54) Method for the Preparation of N-Oxyl Hindered Amine Inhibitors

(71) Applicant: Rohm and Haas Company, Philadelphia, PA (US)

(72) Inventors: Robert Wilczynski, Yardley, PA (US); Muhunthan Sathiosatham, Chalfont, PA (US); Jamie J. Juliette, Houston, TX (US); Lan T.P. Hoang Nguyen, Doylestown, PA (US)

(73) Assignee: Rohm and Haas Company, Philadelphia (US)

(21) Appl. No.: 14/363,968

(22) PCT Filed: Dec. 4, 2012
(86) PCT No.: PCT/US2012/067768

§ 371 Date: Jun. 9, 2014

Related U.S. Application Data
(60) Provisional application No. 61/577,272, filed on Dec. 19, 2011.

(51) Int. Cl. C07D 401/12

(52) U.S. Cl. CPC C07D 401/12
USPC 546/188

(57) ABSTRACT

The present disclosure provides a composition and method for the preparation of N-oxyl hindered amine esters by contacting a compound of Formula (I), wherein R₁ and R₂ are independently an alkyl, with a compound of Formula (II), wherein R₃ and R₄ are independently an alkyl, and n is an integer from 3 to 10, in the presence of a catalyst and a solvent, wherein the catalyst comprises at least one of tin, lithium, zirconium, and hafnium.

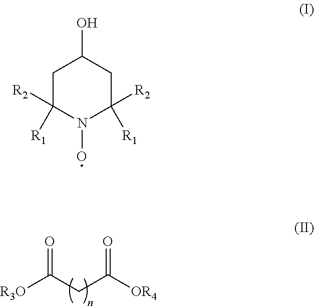

### FIELD OF THE INVENTION

This disclosure relates to an improved method for preparing N-oxyl derivatives of hindered amine esters.

### INTRODUCTION

The preparation of N-oxyl compounds is typically performed by oxidizing a hindered amine with a hydroperoxide in the presence of an oxide catalyst. For example, U.S. Pat. No. 5,218,116 discloses hindered amines oxidized by hydrogen peroxide and a titanium catalyst to produce N-oxyl derivatives.

Russian Patent No. 1,168,556 discloses the preparation of 1-oxyl-2,2,6,6-tetramethylpiperidin-yl esters of carboxylic acids by reacting the corresponding 4-hydroxy compound with a lower alkyl ester of a carboxylic acid in the presence of a tetraalkyl orthotitanate transesterification catalyst in xylene.

U.S. Pat. No. 5,574,163 discloses preparing N-oxyl hindered amine esters by reacting an N-oxyl compound in the presence of a tetraalkyl orthotitanate or a trialkoxy titanium chloride transesterification catalyst and an aliphatic hydrocarbon solvent.

There exists a need for an improved process for preparing N-oxyl hindered amine inhibitors with higher yields, lower cost, and shorter reaction times.

### SUMMARY

In an embodiment, the present disclosure presents a method of preparing a hindered amine inhibitor of Formula III:

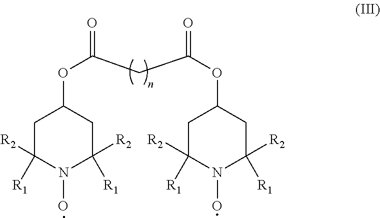

wherein n is an integer from 3 to 10, R₁ and R₂ are independently an alkyl, comprising: contacting a compound of Formula I:

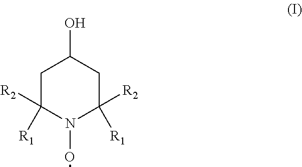

wherein R₁ and R₂ are independently an alkyl,

with a compound of Formula II

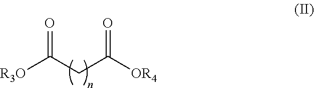

wherein R₃ and R₄ are independently an alkyl, and n is an integer from 3 to 10, in the presence of a catalyst and a solvent, wherein the catalyst comprises at least one of tin, lithium, zirconium, and hafnium.

In an embodiment, the present disclosure presents a composition comprising:

a) a compound of Formula I

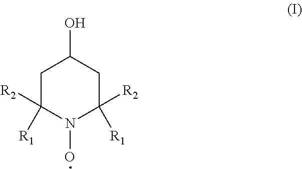

wherein R₁ and R₂ are independently methyl or ethyl;

b) a compound of Formula II

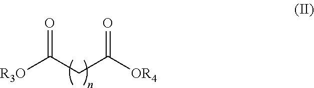

wherein R₃ and R₄ are independently a methyl or ethyl, and n is an integer from 3 to 10;

c) a catalyst comprising at least one of tin, lithium, zirconium, and hafnium; and

d) a solvent.

### DETAILED DESCRIPTION

### Definitions

The numerical ranges in this disclosure are approximate, and thus may include values outside of the range unless otherwise indicated. Numerical ranges include all values from and including the lower and the upper values, in increments of one unit, provided that there is a separation of at least two units between any lower value and any higher value. As an example, if a compositional, physical or other property, such as, for example, molecular weight, etc., is from 100 to 1,000, then all individual values, such as 100, 101, 102, etc., and sub ranges, such as 100 to 144, 155 to 170, 197 to 200, etc., are expressly enumerated. For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure.

### Method

The present disclosure is directed to an improved method for the preparation of N-oxyl hindered amine. The method comprises contacting a compound of Formula I:

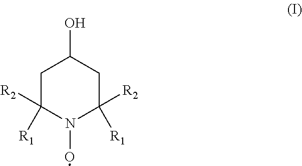

wherein R₁ and R₂ are independently an alkyl, for example methyl or ethyl, with a compound of Formula II

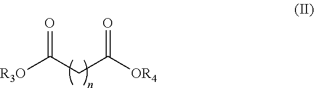

wherein R₃ and R₄ are independently an alkyl, for example methyl or ethyl, and n is an integer from 3 to 10, in the presence of a catalyst comprising at least one of tin, lithium, zirconium, and hafnium, and a solvent. In an embodiment, two equivalents of the compound of Formula I is used to every one equivalent of the compound of Formula II. In an embodiment, contacting a compound of Formula I with a compound of Formula II yields an N-oxyl hindered amine of Formula III, where n, R₁ and R₂ are defined above.

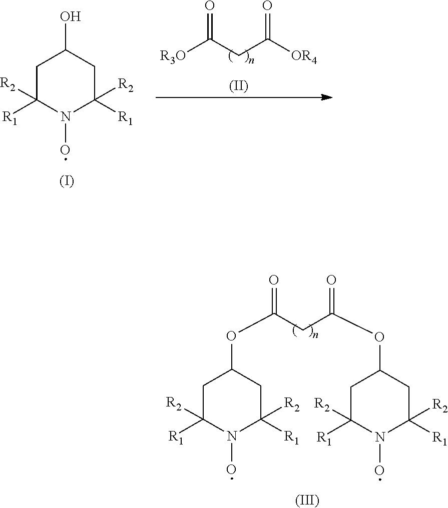

In an embodiment, the composition can include mole ratios of compounds of Formulas I to II that range from 1:1 (to make the half ester of N-oxyl hindered amine with the sebbecate) up to 3:1 (where an excess of N-oxyl hindered amine of Formula I will remain unreacted in the final product but will not be detrimental to the performance of compound of Formula III.

The catalysts of the composition may include barium, magnesium, strontium, calcium, lithium, tin, zirconium, and hafnium compounds. Examples of such catalysts include dialkyltin oxides (e.g, dibutyltin oxide), lithium salts (e.g., LiOH, LiCl), zirconium and hafnium salts. The zirconium and hafnium salts include, but are not limited to, counter-ions such as acetyl acetonate, acetate, acrylate, methacrylate, phenolate, halides (such as choloride, bromide and iodide), and nitrate anions. In one embodiment, the catalysts include barium oxide, magnesium oxide, strontium oxide, calcium oxide, 1,4-diazabicyclo[2,2,2]octane (DABCO) and basic ion exchange resins (such as Amberlyst A26 hydroxide form or Amberlyst A21 free base).

Advantages of the various catalysts include that certain catalysts may be used at lower concentrations than the conventional titanate catalysts, the costs of certain catalysts are lower than the conventional titanate catalysts, and certain catalysts may be more active and yield faster reaction rates than the conventional titanate catalysts.

Suitable solvents include alkanes and cycloalkanes, such as heptane and cyclohexane. In an embodiment, the solvent is aromatic solvents such as toluene.

The solvent may be present in the composition from 5 wt % to 50 wt %, based on the total weight of the composition.

In an embodiment, the composition includes absorbents. Absorbents (e.g., molecular sieves, such as 3A and 4A molecular sieves) may be added to remove the methanol towards the end of the reaction to accelerate the conversion. Absorbent levels may be from 1 wt % to 50 wt %.

In an embodiment, compounds of Formula I and Formula II are combined with a solvent and initially heated to the boiling point of the water/solvent azeotrope under atmospheric pressure or lower, for example from 70 mmHg to 50 mmHg. The solvent may be distilled to effectively dehydrate the reaction mixture by any suitable distillation apparatus. In an embodiment, the distillation apparatus used is a Dean Stark apparatus, which returns the solvent upper layer back into the reaction for 30-60 minutes. The bottom aqueous layer in the Dean Stark receiver is removed via a bottom take-off valve. After the dehydration distillation step, 0.5 to 5 mole percent (mol %) based on the limited reagent of the catalyst may be added to the reaction mixture. The reaction mixture is heated to a temperature range where the distillate received in the Dean stark receiver is an azeotropic mixture between the solvent and methanol by-product from the transesterification reaction. The exact temperature of the reaction mixture is dependent on the solvent used, but is typically from 80° C. to 135° C. The methanol forms a separate lower layer in the Dean stark collection vessel, with the upper solvent layer being returned to the reaction phase. The methanol may be removed and measured for the reaction conversion.

Methanol is soluble in toluene and slightly soluble in heptane. Therefore, a reflux splitter is typically required during all stages of the reaction when toluene is the solvent and during the later stages of the reaction when heptane is used as solvent. The methanol by-product typically forms a separate layer with heptane during first 60-70% of the transesterification reaction. However, during the later stages of the reaction, where small levels of returned methanol can hinder further and complete reaction, the Dean Stark apparatus needs to be replaced by a straight lead distillation unit set at high reflux ratio. This later step allows for efficient removal of the remaining amounts of methanol to help drive the reaction to completion.

After 2 to 8 hours the reaction mixture may be cooled to 60° C. to 70° C., or cooler (e.g., room temperature) and the Dean Stark apparatus removed and replaced with a distillation column equipped with a reflux splitter, set to a reflux to distillate ratio from 75-99 to 25-1, for example 99:1. Another 0.5 to 5 mol % of catalyst (relative to the starting limiting reagent) may be added into the reaction mixt ure and heated to a temperature to cause distillation. After 1-10 hours, the reaction mixture may be cooled 60° C. to 70° C., or cooler (e.g., room temperature). Optionally, a third addition of catalyst at 0.25 to 2.5 mole % (based on the limiting reagent) along with further heating of the reaction mixture along with distillation employing the straight lead distillation unit with high reflux ratio may be applied for another 2 to 10 hours to help drive reaction further to completion. After, the reaction mixture is then cooled to room temperature and allowed to stand for 3 to 24 hours. In an embodiment, the crystals of Formula III which form in the reaction mixture are filtered, washed, and dried in an oven at 50° C. for 12 hours.

### Composition

The present disclosure is directed to a composition comprising a) a compound of Formula I; b) a compound of Formula II; c) a compound comprising at least one of tin, lithium, zirconium, and hafnium; and d) a solvent, wherein Formula I and II are defined above. In an embodiment, the composition comprises two equivalents of Formula I for every one equivalent of Formula II.

In an embodiment, the composition comprises a compound of Formula I where R₁ and R₂ are both methyl groups. In an embodiment, the composition comprises a compound of Formula II where R₃ and R₄ are both methyl and n is from 6 to 10, for example 8.

The composition may further comprise a compound of Formula III:

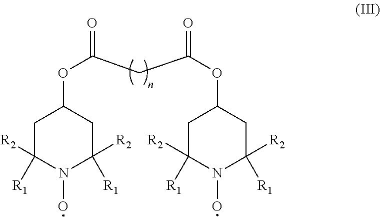

wherein n, R₁ and R₂ are defined above.

The compound comprising at least one of barium, magnesium, strontium, calcium, lithium, tin, zirconium, and hafnium compounds. Examples of such catalysts include dialkyltin oxides (e.g, dibutyltin oxide), lithium salts (e.g., LiOH, LiCl), zirconium and hafnium salts. The zirconium and hafnium salts include, but are not limited to, counter-ions such as acetyl acetonate, acetate, acrylate, methacrylate, phenolate, halides (such as choloride, bromide and iodide), and nitrate anions. In one embodiment, the catalysts include barium oxide, magnesium oxide, strontium oxide, calcium oxide, 1,4-diazabicyclo[2,2,2]octane (DABCO) and basic ion exchange resins (such as Amberlyst A26 hydroxide form or Amberlyst A21 free base).

The compound comprising at least one of tin, lithium, zirconium, and hafnium may be employed from 0.5 to 5 mol %, based on the molar amount of ht elimiting reagent within the reaction composition.

Suitable solvents include alkanes, such as heptane, octane, and aromatic solvents such as toluene. The solvent may be present in the composition from 5 wt % to 50 wt %, based on the total weight of the composition.

In an embodiment, the composition includes absorbents.

### SPECIFIC EMBODIMENTS

### Conversion Testing Method

The reaction conversion is confirmed by ¹H NMR analysis of the reaction mixture, where a small sample (i.e. two drops) is withdrawn from the batch while still warm and homogeneous and is completely dissolved in CDCl₃ solvent. The ¹H NMR analysis of this solution shows reaction progress by measuring the decrease in intensity of methyl ester hydrogen resonances at 3.8 ppm in the ¹H NMR spectrum as compared to methylene hydrogen resonances at 2.5 ppm which are associated with the methylene groups immediately adjacent to the carboxylate functionalities in both the dimethyl sebacate and in the bis-(1-oxyl-2,2,6,6-tetramethylpiperidin-4-yl) sebacate product, which do not change in intensity as reaction occurs. This is because these methylene resonances remain the same intensity and in the same location in the NMR spectrum as the dimethyl sebacate converts to the corresponding bis-(1-oxyl-2,2,6,6-tetramethylpiperidin-4-yl) sebacate product). Due to the paramagnetic nature of the 4-HT, the molecule (as well as this portion of the bis-(1-oxyl-2,2,6,6-tetramethylpiperidin-4-yl) sebacate molecule) is not observed in the NMR spectrum for the reaction solution. Therefore, only the unreacted dimethyl sebacate and the sebacate portion of the final bis-(1-oxyl-2,2,6,6-tetramethylpiperidin-4-yl) sebacate molecule are observed and able to be quantified by this method.

### Inventive Example 1 (IE 1)

### Transesterification Employing Dibutyl Tin Oxide and Heptane

1-oxyl-2,2,6,6-tetramethylpiperidin-4-ol (172.2 grams, 1.0 mole), dimethyl sebacate (115.2 grams, 0.50 mole), and dry heptane (300 ml) is added to a round bottom flask equipped with a Dean Stark receiver. The stirrer is started and the pot solution is heated under atmospheric pressure. The pot contents are heated to a temperature range of 90° C.-105° C., where heptane solvent is distilled into the Dean Stark apparatus, allowing for return of the heptane upper layer back into the pot, for 30-60 minutes. The pot solution is sampled after this dehydration step and analyzed by Karl Fischer titration to ensure the water concentration in the pot is below 0.03 weight % before proceeding further. Once an acceptable water concentration is attained, 2.48 grams (0.01 mole) of dibutyltin oxide, the transesterification catalyst, is charged to the pot. The reaction mixture is heated to 98° C.-102° C. yielding a distillate that comes over into the Dean Stark receiver forming an azeotropic mixture between heptane solvent and methanol by-product from the transesterification reaction. The methanol forms a separate lower layer in the Dean Stark collection vessel, with the upper heptane layer being returned to the reaction phase. This methanol can be removed and measured for the reaction conversion. After 6 hours, 16.6 grams (0.52 mole, of 1 mole expected for complete conversion) of methanol had been removed, which corresponds to 52% conversion of the dimethyl sebacate to bis-(1-oxyl-2,2,6,6-tetramethylpiperidin-4-yl) sebacate

The reaction mixture is cooled to 60° C.-70° C. and the overhead system is changed. The Dean Stark unit is removed and replaced with a 2-plate Oldershaw column equipped with a reflux splitter on top. The reflux splitter is set for a 99:1 reflux:distillate ratio. In addition, 2.48 grams (0.01 mole) of dibutyltin oxide is added and mixed into the reaction mixture and heated to a temperature of 102° C.-104° C. The azeotrope again distills yielding a separate methanol lower layer. After 9 hours (15 hours total), 12.2 grams (0.38 mole) of methanol is removed from the overhead distillate. The total methanol removed corresponds to a 90% conversion of the dimethyl sebacate to the bis-(1-oxyl-2,2,6,6-tetramethylpiperidin-4-yl) sebacate. The reaction mixture is cooled to 60-70° C. and 1.24 grams (0.005 mole) of dibutyltin oxide is added to the reaction mixture.

The reaction mixture is heated to 102° C.-104° C. and maintained at this temperature for 5 hours while also distilling the heptane/methanol azeotrope at the 99:1 reflux ratio. 1.6 grams (0.05 mole) of methanol is collected. Total methanol collected at this point (i.e. after 20 hours total reaction time) equaled 30.4 grams (0.95 mol), which was equivalent to 95% conversion of the dimethyl sebacate. Consistent with this, a ¹H NMR analysis for the reaction solution also showed that 95% of the methyl esters had been displaced within the dimethyl sebacate.

The reaction mixture is cooled to room temperature and allowed to stand overnight. Red crystals formed which are isolated by filtering through a medium filter (Fisher P8; pore size 4-8 μm). The crystalline solids are washed with clean heptane and then placed in a vacuum oven at 50° C. for 12 hours. The solids are then cooled to room temperature and weighed. The isolated yield is 240.1 grams (0.47 mol) which represents 94% isolated yield based on starting materials. The melting point for the solids measured 99.7° C., which corresponds to the reported melting point of 99° C.-101° C. for the non-recrystallized (i.e. crude) bis-(1-oxyl-2,2,6,6-tetramethylpiperidin-4-yl) sebacate product. Inventive Example 2 (IE 2)

### Transesterification Reaction with Zirconium Acetylacetonate and Heptane

1-oxyl-2,2,6,6-tetramethylpiperidin-4-ol (172.2 grams, 1.0 mole), dimethyl sebacate (115.2 grams, 0.50 mole), and dry heptane (300 ml) is added to a round bottom flask equipped with a Dean Stark receiver. The reaction mixture is heated to a temperature range of 90° C.-105° C., in which the heptane solvent is distilled into the Dean Stark apparatus, allowing for return of the heptane upper layer back into the pot, for 30-60 minutes. The reaction mixture is sampled after the dehydration step and analyzed by Karl Fischer titration to ensure the water concentration in the pot was below 0.03 weight % before proceeding further. Once an acceptable water concentration is attained, 4.88 grams (0.01 mole) of zirconium acetylacetonate, the transesterification catalyst, is added to the reaction mixture.

The reaction mixture is heated to 98° C.-102° C. yielding a distillate that comes over into the Dean Stark receiver forming an azeotropic mixture between heptane solvent and methanol by-product from the transesterification reaction. The methanol forms a separate lower layer in the Dean Stark collection vessel, with the upper heptane layer being returned to the reaction phase. This methanol can be removed and measured for the reaction conversion. After 6 hours, 25.3 grams (0.79 mol) of methanol had been removed, which corresponds to 79% conversion of the dimethyl sebacate to the bis-(1-oxyl-2,2,6,6-tetramethylpiperidin-4-yl) sebacate.

The reaction mixture is cooled to 60° C.-70° C. and the overhead system is changed. The Dean Stark unit is removed and replaced with a 2-plate Oldershaw column equipped with a reflux splitter on top. The reflux splitter is set for a 99:1 reflux:distillate ratio. 4.88 grams (0.01 mole) of the zirconium acetylacetonate catalyst is added to the reaction mixture and heated to 102° C.-104° C. The azeotrope distilled over giving a separate methanol lower layer. After 6 hours (12 hours total) 5.5 grams (0.17 mole) of methanol is removed from the overhead distillate. The total methanol removed corresponds to a 96% conversion of the dimethyl sebacate to the bis-(1-oxyl-2,2,6,6-tetramethylpiperidin-4-yl) sebacate. An NMR analysis of the reaction solution suggested that the dimethyl sebacate was only 93% converted. The reaction mixture was cooled down to 60-70° C. and 2.44 grams (0.005 mol) of zirconium catalyst is added to the reaction solution.

The reaction mixture is again heated to 102° C.-104° C. and maintained at this temperature for another 3 hours while also distilling the heptane/methanol azeotrope at the 99:1 reflux ratio. In this 3 hour period 1.5 grams of methanol is collected. The total methanol collected after 15 hours of total reaction time equaled 31.6 grams (0.99 mol), which was equivalent to 99% conversion of the dimethyl sebacate. A ¹H NMR analysis for the reaction solution showed that 94% of the methyl esters had been displaced within the dimethyl sebacate.

The reaction mixture is cooled to room temperature and allowed to stand overnight. Red crystals formed which are isolated by filtering through a medium filter (Fisher P8; pore size 4-8 μm). The crystalline solids are washed with heptane and then placed in a vacuum oven at 50° C. for 12 hours. The solids are then cooled to room temperature and weighed. The isolated yield was 247.8 g (0.485 mol) which represents 97% isolated yield based on starting reactants. The melting point for the solids measured 98.8° C.

### Inventive Example 3 (IE 3)

### Transesterification Employing Hafnium Acetylacetonate and Heptane

1-oxyl-2,2,6,6-tetramethylpiperidin-4-ol (172.2 grams, 1.0 mole), dimethyl sebacate (115.2 grams, 0.50 mole), and dry heptane (300 ml) is added to a round bottom flask equipped with a Dean Stark receiver. The reaction mixture is heated to 90° C.-105° C., where heptane solvent is distilled into the Dean Stark apparatus, allowing for return of the heptane upper layer back into the pot, for 30-60 minutes. Once the water concentration is below 0.03 weight %, 5.75 grams (0.01 mole) of Hafnium Acetylacetonate, the transesterification catalyst, is added to the reaction mixture.

The reaction mixture is heated to 98° C.-102° C. yielding a distillate that comes over into the Dean Stark receiver as an azeotropic mixture between heptane solvent and methanol by-product from the transesterification reaction. The methanol forms a separate lower layer in the Dean Stark collection vessel, with the upper heptane layer being returned to the reaction phase. After 6 hours, 27.5 grams (0.86 mole) of methanol had been removed, which corresponds to 86% conversion of the dimethyl sebacate to the bis-(1-oxyl-2,2,6,6-tetramethylpiperidin-4-yl) sebacate product.

The reaction mixture is cooled to 60° C.-70° C. and the overhead system is changed. The Dean Stark unit is removed and replaced with a 2-plate Oldershaw column equipped with a reflux splitter on top. The reflux splitter was set for a 99:1 reflux:distillate ratio (reflux=distillate returned to the top of the Oldershaw column). 5.75 grams (0.01 mole) of hafnium acetylacetonate is added and heated to 102° C.-104° C. Under these conditions the azeotrope again distilled over giving a separate methanol lower layer. After another 6 hours (12 hours total), 2 grams (0.06 mole) of methanol is removed from the overhead distillate. The total methanol removed corresponds to a 92% conversion of the dimethyl sebacate to the bis-(1-oxyl-2,2,6,6-tetramethylpiperidin-4-yl) sebacate. The reaction mixture was cooled to 60° C.-70° C. and 2.87 grams (0.005 mole) of hafnium acetylacetonate is added to the reaction mixture.

The reaction mixture is heated to 102° C.-104° C. and maintained at this temperature for another 8 hours while also distilling the heptane/methanol azeotrope at the 99:1 reflux ratio. In this 8 hour period no more methanol appeared to be collected. A ¹H NMR analysis for the reaction solution was consistent in showing that approximately 92% of the methyl esters had been displaced within the dimethyl sebacate.

The reaction mixture is then cooled to room temperature and allowed to stand overnight. Red crystals formed which were isolated by filtering through a medium filter (Fisher P8; pore size 4-8 μm). The crystalline solids are washed with heptane and then placed in a vacuum oven at 50° C. for 12 hours. The solids are then cooled to room temperature and weighed. The isolated yield was 252.8 grams (0.495 mol) which represents 99% isolated yield based on starting materials. The melting point for the solids measured 96.4° C. Importantly, this melting point is much closer to that for the bis-(1-oxyl-2,2,6,6-tetramethylpiperidin-4-yl) sebacate than that for both the dimethyl sebacate (a liquid at room temperature) and 4-HT (mp=80° C.), indicating that the solids are of nearly equal purity to the products made in our other examples.

### Inventive Example 4 (IE 4)

Transesterification Employing Zirconium Acetylacetonate and Octane

1-oxyl-2,2,6,6-tetramethylpiperidin-4-ol (172.2 grams, 1.0 mole), dimethyl sebacate (115.2 grams, 0.50 mole), and dry octane (300 ml) is added to a round bottom flask fitted with a Dean Stark receiver. The reaction mixture is heated to a temperature range of 120° C.-130° C., where octane solvent is distilled into the Dean Stark apparatus, allowing for return of the octane upper layer back into the pot, for 30-60 minutes. Once the water concentration is below 0.03 weight %, 4.88 grams (0.01 mole) of zirconium acetylacetonate, the transesterification catalyst, is added to the reaction mixture.

The reaction mixture is heated to 110° C.-120° C. yielding a distillate that comes over into the Dean Stark receiver as an azeotropic mixture between heptane solvent and methanol by-product from the transesterification reaction. The methanol forms a separate lower layer in the Dean Stark collection vessel, with the upper octane layer being returned to the reaction phase. After 2.5 hours, 22.3 grams (0.70 mol) of methanol had been removed, which corresponds to 70% conversion of the dimethyl sebacate to the bis-(1-oxyl-2,2,6,6-tetramethylpiperidin-4-yl) sebacate.

The reaction mixture is cooled to 60° C.-70° C. and the overhead system is changed. The Dean Stark unit is removed and replaced with a 2-plate Oldershaw column equipped with a reflux splitter on top. The reflux splitter is set for a 99:1 reflux:distillate ratio. 4.88 grams (0.01 mole) of the zirconium acetylacetonate catalyst is added to the reaction mixture and heated to 120° C.-130° C. Under these conditions the azeotrope again distilled over giving a separate methanol lower layer. After another 1.5 hours (4 hours total), 8.1 grams (0.25 mole) of methanol is removed from the overhead distillate. The total methanol removed corresponds to a 95% conversion of the dimethyl sebacate to the bis-(1-oxyl-2,2,6,6-tetramethylpiperidin-4-yl) sebacate. An NMR analysis of the reaction solution suggested that the dimethyl sebacate was only 90% converted. The reaction mixture is cooled down to 60° C.-70° C. and 2.44 grams (0.005 mol) of zirconium catalyst is added to the reaction mixture.

The reaction mixture is heated to 120° C.-130° C. and maintained at this temperature for 2 hours while distilling the octane/methanol azeotrope at the 99:1 reflux ratio. In this 2 hour period 1.3 grams of methanol is collected. Total methanol collected at this point (i.e. after 6 hours total reaction time) equaled 31.7 grams (0.99 mol), which is equivalent to 99% conversion of the dimethyl sebacate. A ¹H NMR analysis for the reaction solution showed that 95% of the methyl esters had been displaced within the dimethyl sebacate.

The reaction mixture is cooled to room temperature and allowed to stand overnight. Red crystals formed which are isolated by filtering through a medium filter (Fisher P8; pore size 4-8 μm). The crystalline solids are washed with heptane and then placed in a vacuum oven at 50° C. for 12 hours. The solids are then cooled to room temperature and weighed. The isolated yield was 247.8 g (0.485 mol) which represents 97% isolated yield based on starting reactants. The melting point for the solids measured 98.8° C.

### Comparative Example 1 (CE 1)

Transesterification Employing Tetra 2-Ethyl Hexyl Titanate and Heptane

1-oxyl-2,2,6,6-tetramethylpiperidin-4-ol (172.2 grams, 1.0 mole), dimethyl sebacate (115.2 grams, 0.50 mole), and dry heptane (300 ml) is added to a round bottom flask equipped with a Dean Stark receiver. The stirrer was started and the pot solution is heated to 90° C.-105° C. under atmospheric pressure. The heptane solvent is distilled into the Dean Stark apparatus, allowing for return of the heptane upper layer back into the pot, for 30-60 minutes. The reaction mixture is sampled after the dehydration step and analyzed by Karl Fischer titration to ensure the water concentration in the pot is below 0.03 weight % before proceeding further. Once an acceptable water concentration was attained, 5.64 grams (0.01 mole) of tetra-2-ethylhexyloxy titanate, the transesterification catalyst, is charged to the pot.

The reaction mixture is heated to 98° C.-102° C. yielding an azeotropic mixture between heptane solvent and methanol by-product from the transesterification reaction. The methanol forms a separate lower layer in the Dean Stark collection vessel, with the upper heptane layer being returned to the reaction phase. After 6 hours, 20.8 grams (0.65 mol) of methanol had been removed, which corresponds to 65% conversion of the dimethyl sebacate to the bis-(1-oxyl-2,2,6,6-tetramethylpiperidin-4-yl) sebacate.

The reaction mixture is cooled to 60° C.-70° C. and the overhead system is changed. The Dean Stark unit is removed and replaced with a 2-plate Oldershaw column equipped with a reflux splitter on top. The reflux splitter is set for a 99:1 reflux:distillate ratio. 5.64 grams (0.01 mole) of the tetra-2-ethylhexyloxy titanate catalyst is added into the reaction mixture and heated to 102° C.-104° C. The azeotrope distilled yielding a separate methanol lower layer. After another 6 hours (12 hours total), 9.3 grams (0.29 mole) of methanol is removed from the overhead distillate. The total methanol removed corresponds to a 94% conversion of the dimethyl sebacate to the bis-(1-oxyl-2,2,6,6-tetramethylpiperidin-4-yl) sebacate. The reaction mixture is cooled down to 60° C.-70° C. and 2.82 grams (0.005 mol) of titanate catalyst is added to the reaction solution.

The reaction mixture is heated to 102° C.-104° C. and maintained at this temperature for another 3 hours while also distilling the heptane/methanol azeotrope at the 99:1 reflux ratio. In this 3 hour period another 1.6 grams (0.05 mol) of methanol is collected. Total methanol collected at this point (i.e. after 15 hours total reaction time) equaled 31.6 grams (0.99 mol), which is equivalent to 99% conversion of the dimethyl sebacate. Consistent with this, a ¹H NMR analysis for the reaction solution also showed that 99% of the methyl esters had been displaced within the dimethyl sebacate.

The reaction mixture is then cooled to room temperature and allowed to stand overnight. Red crystals formed which are isolated by filtering this suspension of solids through a medium filter (Fisher P8; pore size 4-8 μm). The crystalline solids are washed with heptane and then placed in a vacuum oven at 50° C. for 12 hours. The solids are then cooled to room temperature and weighed. The isolated yield was 232.5 g (0.455 mol) which represents 91% isolated yield based on starting materials. The melting point for the solids measured 99.1° C.

Table 1 presents a summary of Inventive Examples 1-4 and Comparative Example 1.

TABLE 1
Summary of IE 1-IE 4 and CE 1

| | IE 1 | IE 2 | IE 3 | IE 4 | CE 1 |
|---|---|---|---|---|---|
| Solvent | heptane | heptane | heptane | octane | heptane |
| 1-oxyl-2,2,6,6- | 1.0 | 1.0 | 1.0 | 1.0 | 1.0 |
| tetramethyl piperidin-4-ol | | | | | |
| (mol) | | | | | |
| Dimethyl sebacate | 0.5 | 0.5 | 0.5 | 0.5 | 0.5 |
| (mol) | | | | | |
| Zirconium | | 0.025 | | 0.025 | |
| acetylacetonate (mol) | | | | | |
| Dibutyltin oxide | 0.025 | | | | |
| (mol) | | | | | |
| Tetra 2-ethyl hexyl | | | | | 0.025 |
| titanate (mol) | | | | | |
| Hafnium acetylacetonate | | | 0.025 | | |
| (mol) | | | | | |
| Conversion (mol %) | 94% | 97% | 99% | 97% | 91% |

Table 1 demonstrates higher yields were obtained with all other catalysts as compared to that obtained employing the conventional titanate catalyst.

1. A method of preparing a hindered amine inhibitor of Formula III: 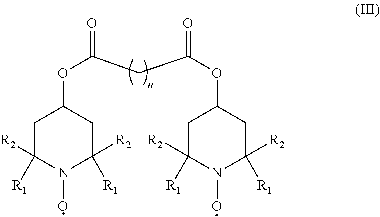
wherein n is an integer from 3 to 10, R₁ and R₂ are independently an alkyl, comprising:
(A) contacting a compound of Formula I: 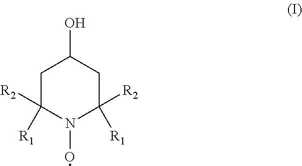
wherein R₁ and R₂ are independently an alkyl,
with a compound of Formula II 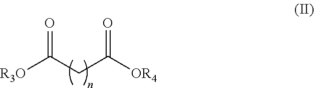
wherein R₃ and R₄ are independently an alkyl, and n is an integer from 3 to 10,
in the presence of a catalyst and a solvent to form a reaction mixture,
wherein the catalyst comprises at least one of tin, lithium, zirconium, and hafnium;
(B) heating the reaction mixture to a temperature from 80° C. to 135° C. and then cooling the reaction mixture to a temperature of 70° C. or cooler;
(C) heating the reaction mixture a second time to a temperature to cause distillation and then cooling the reaction mixture to a temperature of 70° C. or cooler; and
(D) optionally, heating the reaction mixture a third time to a temperature to cause distillation and then cooling the reaction mixture to room temperature.

2. The method of claim 1 wherein R₁-R₄ are methyl groups and n is from 6 to 8.

3. The method of claim 1 further comprising the step of employing a reflux-to-distillate ratio of at least 99 to 1.

4. The method of claim 1 wherein the catalyst is dialkyltin oxide.

5. The method of claim 1 wherein the catalyst is lithium hydroxide.

6. The method of claim 1 wherein the catalyst is zirconium acetyl acetonate.

7. The method of claim 1 wherein the catalyst is hafnium acetylacetonate.

8. A composition comprising:
a) a compound of Formula I 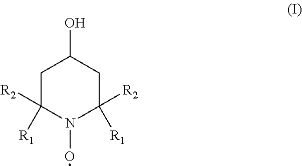
wherein R₁ and R₂ are independently methyl or ethyl;
b) a compound of Formula II 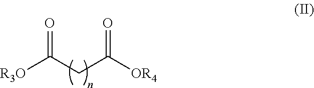
wherein R₃ and R₄ are independently a methyl or ethyl, and n is an integer from 3 to 10;
c) a catalyst comprising at least one of tin, lithium, zirconium, and hafnium; and
d) a solvent,
wherein the mole ratio of the compound of Formula I to that of the compound of Formula (II) is from 1:1 to 3:1, and the amount of catalyst is form 0.5 to 5 mol % based on the molar amount of the limitation reagent of the composition.

9. The composition of claim 8 comprising twice as many equivalents of the compound of Formula I than the compound of Formula II.

10. The composition of claim 8 wherein R₁-R₄ are methyl groups and n is from 6 to 8.

11. The composition of claim 8 further comprising a hindered amine inhibitor of Formula III: 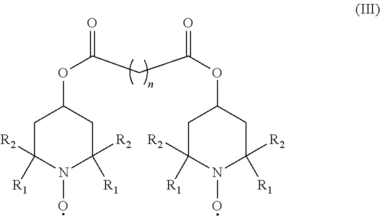
wherein n is an integer from 3 to 10, R₁ and R₂ are independently an alkyl radical.

12. (canceled)
